(19) United States
(12) Patent Application Publication
(10) Pub. No.: US 2008/0254147 A1
(43) Pub. Date: Oct. 16, 2008

(54) METHOD OF PROVIDING HEMOSTASIS IN ANTI-COAGULATED BLOOD

(75) Inventors: Raymond J. Huey, Orange, CT (US); Giacomo Basadonna, Haddam, CT (US)

Correspondence Address: MICHAUD-DUFFY GROUP LLP, 306 INDUSTRIAL PARK ROAD, SUITE 206, MIDDLETOWN, CT, 06457, US

(73) Assignee: Z-MEDICA CORPORATION, Wallingford, CT (US)

(21) Appl. No.: 12/101,346

(22) Filed: Apr. 11, 2008

Related U.S. Application Data
(63) Continuation-in-part of application No. 12/101,336, filed on Apr. 11, 2008.
(60) Provisional application No. 60/923,416, filed on Apr. 13, 2007.

(51) Int. Cl. A61K 33/06; A61P 7/04

(52) U.S. Cl. 424/684

(57) ABSTRACT

In a method of clotting blood in which the blood exhibits a reduced tendency to clot and may be from a person undergoing an anticoagulant therapy or having type A or B hemophilia or von Willebrand disease, a therapeutically effective amount of a composition comprising clay as the active ingredient is administered to a wound from which the blood emanates. Upon contacting the blood, this clay, which may be kaolin, bentonite, or any type of layered clay, causes the blood to clot. In a method of arresting blood flowing from a wound, a therapeutically effective amount of a composition comprising clay as the active ingredient is administered to the bleeding wound. In this method, the blood has a reduced tendency to clot and may be from a person undergoing an anticoagulant therapy or having at least one of hemophilia A or B or von Willebrand disease.

### CROSS REFERENCE TO RELATED APPLICATION

This application is a continuation-in-part of and claims the benefits of [Attorney Docket No. 6989-0087-1], which is filed concurrently herewith, which claims the benefits of U.S. Provisional Patent Application Ser. No. 60/923,416, filed on Apr. 13, 2007, the contents of all of the foregoing applications being incorporated by reference herein in their entireties.

### TECHNICAL FIELD

The present invention relates generally to methods of providing hemostasis in blood that is resistant to normal clotting functions and, more particularly, to methods of providing hemostasis in patients having compromised blood clotting functions due to the use of anticoagulant compositions or due to deficiencies in factors that contribute to clotting abilities.

### BACKGROUND OF THE PRESENT INVENTION

Blood is a liquid tissue that includes red cells, white cells, corpuscles, and platelets dispersed in a liquid phase. The liquid phase is plasma, which includes acids, lipids, solublized electrolytes, and proteins. Some proteins and other substances in the plasma are collectively known as clotting factors (indicated by Roman numerals) and function together to promote the coagulation of blood. The proteins are suspended in the liquid phase. One particular protein suspended in the liquid phase is fibrinogen.

Anticoagulant drugs are typically prescribed to individuals with increased tendencies for thrombosis, which is the formation of clots in the blood, or as prophylaxis in individuals who have pre-existing blood clots to reduce the risks of embolism. These drugs are also indicated for the long-term anticoagulation treatment of patients having certain kinds of surgery, heart disease, following stent placement, valve replacement, atrial fibrillation, and the like.

One such anticoagulant drug is warfarin, which is a synthetic derivative of 4-hydroxycoumarin and which decreases the natural abilities of blood to coagulate by interfering with the hepatic synthesis of vitamin K-dependent clotting factors, particularly those indicated as Factors II, VII, IX, and X. It also interferes with the regulatory factors protein C, protein S, and protein Z. Other proteins not involved in blood clotting such as osteocalcin and matrix Gla protein may also be affected.

Warfarin is typically used by individuals suffering from atrial fibrillation to reduce the incidence of stroke, thromboembolism, complications associated with cardiac valve replacement, myocardial infarction, and the like. The degree of anticoagulation in an individual undergoing warfarin therapy is determined by the international normalized ratio (INR) of the blood. A normal INR range is 0.8 to 1.2, whereas individuals taking warfarin typically have an INR target range of 2.0 to 3.0. These individuals generally have difficulty in achieving hemostasis after experiencing a wound resulting from trauma (e.g., from an accident or a medical procedure).

Several adverse effects have been noted with regard to individuals undergoing warfarin therapy. Such adverse effects include, but are not limited to, paresthia, headache, joint and/or muscle pain, shortness of breath, swelling, weakness, hypotension, jaundice, fever, hepatitis, alopecia, elevation of liver enzymes, and hemorrhage (bleeding). Hemorrhage is the most common and dangerous complication associated with the regular use of warfarin and occurs in about 2% to about 5% of treated patients with a significant increase in hospitalization and associated costs. The hemorrhage may be from any tissue or organ and may be fatal or non-fatal. Hemorrhage can also be exacerbated by certain vascular defects, abnormalities in the blood, or deficiencies of one or more of the coagulation factors.

Another anticoagulant drug with which the present invention can be used is clopidogrel, which is an antiplatelet agent used in the treatment of coronary artery disease, peripheral vascular disease, and cerebrovascular disease. Clopidogrel works by blocking the adenosine diphosphate (ADP) receptor on platelet cell membranes, which operates to facilitate platelet aggregation in the blood, thereby inhibiting the platelet aggregation by blocking activation of the glycoprotein IIb/IIIa pathway. Clopidogrel is indicated for the prevention of vascular ischaemic events in patients with symptomatic atherosclerosis, acute coronary syndrome, in conjunction with aspirin therapy to prevent thromboembolism after the placement of an intracoronary stent, and the like. Adverse effects include hemorrhage.

Hemorrhage can also occur as the result of traumatic injury irrespective of whether or not the hemorrhaging individual is undergoing warfarin therapy or clopidogrel therapy. When a hemorrhage occurs as a result of trauma and the blood is normal (i.e., not significantly deficient in any component that would alter its ability to clot or not subject to anticoagulant drugs), hemostasis is initiated normally. Hemostasis is the arrest of blood flow from an injured blood vessel and requires the combined functions of the vascular, platelet, and plasma factors. In initiating hemostasis in response to trauma, the physiologic process of thrombosis begins. In thrombosis, the platelets aggregate and/or the fibrinogen reacts with water and thrombin (an enzyme) to form fibrin, which is insoluble in blood and which polymerizes to form the clots.

When hemorrhage occurs as the result of traumatic injury in individuals undergoing warfarin therapy or clopidogrel therapy (or taking some other anticoagulating drug), the ability of the blood to experience normal clotting functions is compromised. This lack of normal clotting functions may prove to be problematic during the course of an attempted emergency treatment of the individual. For example, a caregiver at an accident scene may be unaware that an injured individual may be taking warfarin or clopidogrel and may attempt to provide normal medical treatment, the effects of which may have limited efficacy due to the individual's lack of clotting ability. Treatment of an individual known to be undergoing such therapies via planned surgery, on the other hand, may be less problematic but still pose problems for the persons performing the surgery because the effect of warfarin or clopidogrel has to be reversed.

Hemorrhage can also occur as the result of hemophilia. Hemophilia is the name for several hereditary genetic illnesses that impair the ability of a body to control bleeding. Various types of hemophilia exist. Hemophilia A, the most common form of hemophilia, is a blood clotting disorder caused by a mutation of the Factor VIII gene, which leads to a deficiency in Factor VIII. Inheritance is X-linked recessive; thus, males are affected (1 in 10,000) while females are carriers or very rarely display a mild phenotype. Hemophilia B, the second most common form, is a blood clotting disorder caused by a mutation of the Factor IX gene, which may indicate a deficiency in Factor IX. Hemophilia (all types) affects about 18,000 people in the United States. Each year, about 400 babies are born with the disorder. Patients with hemophilia may bleed for a longer time than others after an injury or accident. They also may bleed internally, especially in the joints (knees, ankles, and elbows).

Hemorrhage can also occur as the result of von Willebrand disease. Von Willebrand disease is the most common hereditary coagulation abnormality described in humans, although it can also be acquired as a result of other medical conditions. It arises from a qualitative or quantitative deficiency of von Willebrand factor (vWF), a multimeric protein that is required for platelet adhesion. The vWF factor is present in blood plasma and produced constitutively in endothelium (in the Weibel-Palade bodies), megakaryocytes (α-granules of platelets), and subendothelial connective tissue. Von Willebrand factor is not an enzyme and therefore has no catalytic activity. Its primary function is binding to other proteins, particularly Factor VIII, and it is important in platelet adhesion to wound sites.

Von Willebrand factor binds to cells and molecules in a number of different scenarios. These scenarios include, but are not limited to: (a) Factor VIII is bound to vWF whilst inactive in circulation, the Factor VIII degrades rapidly when not bound to vWF, and the Factor VIII is released from vWF by the action of thrombin; (b) vWF binds to collagen, e.g., when it is exposed in endothelial cells due to damage occurring to the blood vessel; (c) vWF binds to platelet gplb when it forms a complex with gpIX and gpV (occurs under all circumstances, but is most efficient under high shear stress (i.e., rapid blood flow in narrow blood vessel)); and (d) vWF binds to other platelet receptors when they are activated, e.g., by thrombin (i.e., when coagulation has been stimulated).

There are three types of hereditary von Willebrand disease, namely, Types I, II, and III. Types I and II are considered herein to be mild. In the mild form, a ristocetin co-factor is decreased and different levels of von Willebrand disease multimers are depleted. Type III is considered herein to be severe. In severe von Willebrand disease, only less than 10% expression of factor VIII is present and no detectable level of von Willebrand factor is present.

The various types of von Willebrand disease present varying degrees of bleeding tendency. In any form, bruising, nosebleeds, heavy menstrual periods (in women), and blood loss during childbirth (which is rare) may occur. Also, internal bleeding or joint bleeding may also occur. This type of bleeding is generally only in the severe form of von Willebrand disease and is rare. Particularly with regard to the severe form, death may occur.

Based on the foregoing, it is a general object of the present invention to provide methods of facilitating hemostasis in individuals undergoing warfarin therapy, clopidogrel therapy, or being deficient in certain clotting factors that overcome or improve upon the prior art, such methods being in response to trauma sustained either as a result of an accident or an intentionally inflicted wound.

### SUMMARY OF THE PRESENT INVENTION

In one aspect, the present invention is directed to a method of clotting blood. The blood exhibits a reduced tendency to clot (compared to normal blood) and may be from a person undergoing an anticoagulant therapy or having type A or B hemophilia or von Willebrand disease. In the method a therapeutically effective amount of a composition comprising zeolite as the active ingredient is administered to a wound from which the blood emanates. Upon contacting the blood, the zeolite causes the blood to clot.

In another aspect, the present invention is directed to a method of arresting blood flowing from a wound. The method comprises the step of administering a therapeutically effective amount of a composition comprising zeolite as the active ingredient to the bleeding wound. The blood has a reduced tendency to clot (compared to normal blood) may be from a person undergoing an anticoagulant therapy or having at least one of hemophilia A or B or von Willebrand disease.

In another aspect, the present invention is directed to a method of facilitating the formation of blood clots. In the method, blood treated with an anticoagulant composition, being deficient in either Factor VIII or Factor IX, and/or being deficient in von Willebrand factor is provided and contacted with a negatively charged surface. Upon contacting the blood with the negatively charged surface, a clotting mechanism is initiated.

In another aspect, the present invention is directed to another method of clotting blood in which the blood exhibits a reduced tendency to clot and may be from a person undergoing an anticoagulant therapy or having type A or B hemophilia or von Willebrand disease. In this method, a therapeutically effective amount of a composition comprising clay as the active ingredient is administered to a wound from which the blood emanates. Upon contacting the blood, this clay, which may be kaolin, bentonite, or any type of layered clay, contributes to the clotting of the blood.

In another aspect, the present invention is also directed to a method of arresting blood flowing from a wound in which the method comprises the step of administering a therapeutically effective amount of a composition comprising clay (e.g., kaolin, bentonite, or a layered clay) as the active ingredient to the bleeding wound. The blood has a reduced tendency to clot may be from a person undergoing an anticoagulant therapy or having at least one of hemophilia A or B or von Willebrand disease.

### DETAILED DESCRIPTION OF THE PREFERRED EMBODIMENT

Disclosed herein are methods for delivering hemostatic materials to interface regions of tissue and blood vessels to promote the clotting of blood and to limit the degree of bleeding in individuals having coagulation disorders. As used herein, the term “coagulation disorder” refers to an inability or reduced ability of blood to produce clots. The methods generally comprise stopping bleeding that results from trauma (e.g., from unintentional wounds as well as intentional wounds such as those resulting from surgical procedures) to tissue or organs in individuals undergoing anticoagulant drug therapy. Anticoagulant drugs with which the methods described herein may be used include, but are not limited to, warfarin and other derivatives of 4-hydroxycoumarin (e.g., coumarin-based compositions), clopidogrel and derivatives thereof (e.g., clopidogrel-based compositions), and the like. One exemplary hemostatic material that can be used with the methods of the present invention is zeolite.

As used herein, the term “zeolite” refers to a crystalline form of aluminosilicate having the ability to be dehydrated without experiencing significant changes in the crystalline structure. The zeolite typically includes one or more ionic species such as, for example, calcium and sodium moieties. In zeolites containing calcium and sodium, the calcium portion contains crystals that are about 5 angstroms in size, and the sodium portion contains crystals that are about 4 angstroms in size. The preferred molecular structure of the zeolite is an “A-type” crystal, namely, one having a cubic crystalline structure that defines round or substantially round openings. In its original state, zeolite is negatively charged, which means it has a propensity for attracting positively charged ions.

Zeolites for use in the disclosed applications may be naturally occurring or synthetically produced. Numerous varieties of naturally occurring zeolites are found as deposits in sedimentary environments as well as in other places. Naturally occurring zeolites that may be applicable to the compositions described herein include, but are not limited to, analcite, chabazite, heulandite, natrolite, stilbite, and thomosonite. Synthetically produced zeolites that may also find use in the compositions and methods described herein are generally produced by processes in which rare earth oxides are substituted by silicates, alumina, or alumina in combination with alkali or alkaline earth metal oxides.

The zeolite may be mixed with or otherwise used in conjunction with other materials. These materials may be used as fillers or inert ingredients with the zeolite. Preferably, these materials have the ability to be dehydrated without significant changes in crystalline structure. Such materials include, but are not limited to, magnesium sulfate, sodium metaphosphate, calcium chloride, dextrin, polysaccharides, combinations of the foregoing materials, and hydrates of the foregoing materials. Clays, diatomaceous earth, bioactive glass, chitosan, polymeric materials, and combinations of the foregoing may also be mixed with the zeolite. The present invention is not limited in this regard, however, as other materials may be used in conjunction with the zeolite.

The zeolite may be administered in any suitable form. Suitable forms include, but are not limited to, particles, beads, pellets, chips, flakes, powders, pastes, gels, combinations of the foregoing, and the like.

In effecting hemostasis at a wound site in an individual undergoing warfarin or clopidogrel therapy (or in any individual having a coagulation disorder), the zeolite is administered in a therapeutically effective amount utilizing any suitable delivery mechanism. A therapeutically effective amount is any amount that is capable of causing the anticoagulated blood of the individual to sufficiently clot. If the zeolite is in the form of loose particles such as pellets, beads, or the like, the zeolite can be poured or otherwise placed directly onto the wound site. Loose powder having sufficient fluidity can also be poured or placed directly onto the wound site. If the zeolite is in the form of a paste, e.g., suspended in a gel carrier, the zeolite can be spread or smeared topically over the wound, or it can be applied to bandages, gauze, pads, or other like materials and used to dress the wound. Furthermore, sponges and cloths into which the zeolite is impregnated or otherwise incorporated may be applied to or even packed into the wound.

Another exemplary hemostatic material that can be used with the methods of the present invention is clay. Clays that may be used include layered clays such as kaolin or kaolinite. The present invention is not limited to layered clays, as non-layered clays may be used in place of or in combination with layered clays. Also, the present invention is not limited to kaolin, as other clays (for example, bentonite clays) may be used in place of or in combination with kaolin.

As used herein, the term “clay” refers to a crystalline form of hydrated aluminum silicate. The crystals of clay are irregularly shaped and insoluble in water. The combination of some types of clay with water may produce a mass having some degree of plasticity. Depending upon the type of clay, the combination thereof with water may produce a colloidal gel having thixotropic properties.

As used herein, the term “kaolin” refers to a soft, earthy aluminosilicate clay (and, more specifically, to a dioctahedral phyllosilicate clay) having the chemical formula Al₂Si₂O₅(OH)₄. Kaolin is a naturally occurring layered silicate mineral having alternating tetrahedral sheets and octahedral sheets of alumina octahedra linked via the oxygen atoms of hydroxyl groups. Kaolin comprises about 50% alumina, about 50% silica, and trace impurities.

More preferably, the clay is Edgar's plastic kaolin (hereinafter “EPK”), which is a water-washed kaolin clay that is mined and processed in and near Edgar, Fla. Edgar's plastic kaolin has desirable plasticity characteristics, is castable, and when mixed with water produces a thixotropic slurry.

As with the zeolite, the kaolin or other clay may be mixed with or otherwise used in conjunction with other materials. Such materials include, but are not limited to, magnesium sulfate, sodium metaphosphate, calcium chloride, dextrin, combinations of the foregoing materials, and hydrates of the foregoing materials.

Various materials may be mixed with, associated with, or incorporated into the kaolin to maintain an antiseptic environment at the wound site or to provide functions that are supplemental to the clotting functions of the clay. Exemplary materials that can be used include, but are not limited to, pharmaceutically-active compositions such as antibiotics, antifungal agents, antimicrobial agents, anti-inflammatory agents, analgesics, antihistamines (e.g., cimetidine, chloropheniramine maleate, diphenhydramine hydrochloride, and promethazine hydrochloride), compounds containing silver or copper ions, combinations of the foregoing, and the like. Other materials that can be incorporated to provide additional hemostatic functions include ascorbic acid, tranexamic acid, rutin, and thrombin. Botanical agents having desirable effects on the wound site may also be added.

It is believed that the cellular clotting mechanism of clay activates certain contact factors when applied to blood. More specifically, it is believed that kaolin (particularly EPK) initiates mechanisms by which water in blood is absorbed to facilitate clotting functions.

The kaolin may be administered in any suitable form. In one suitable form, the kaolin is administered via a gauze. More particularly, the kaolin (or other clay) is impregnated into a gauze substrate. The kaolin is coated onto the gauze substrate using any suitable method (e.g., by being dispersed in a slurry into which the gauze substrate is dipped, by being sprayed onto the substrate, or the like). The gauze substrate may be any suitable woven or non-woven fibrous material including, but not limited to, cotton, silk, wool, plastic, cellulose, rayon, polyester, combinations of the foregoing, and the like. The present invention is not limited to woven or non-woven fibrous materials as the gauze substrates, however, as felts and the like are also within the scope of the present invention.

In each of the Examples provided below, human plasma was obtained from two or more patients affected by one single studied condition. The human plasma was obtained from George King Bio-Medical, Inc., Overland Park, Kans. In each of the Examples, results are shown as a mean plus or minus the standard deviation. Student t test was performed as statistical analysis and p<0.05 was considered as significant.

### Example 1

### Use of Zeolite to Treat Human Plasma from Patients Undergoing Warfarin Therapy

Human plasma was obtained from patients treated with Coumadin® (a brand of warfarin) and having INR levels of 1.9, 3.6, and 5.3. Plasma from three patients per INR level was analyzed. The plasma was divided in 2 groups (Control and Study 1) and was tested in vitro in a modified PT manual test. For the test, 0.25 ml of plasma was incubated with 25% dilution in 0.9% saline of Simplastin Excel (thromboplastin reagent, available from Biomerieux, Durham, N.C.). Zeolite material was added to the Study 1 group samples. Results are shown in Table 1.

TABLE 1
Table 1: Zeolite-treated plasma clots significantly faster than
untreated controls.

| | Time to clot: |
|---|---|
| | seconds |
| INR 1.9 | |
| Control n = 2 | 172.5 ± 10.6 |
| Study 1 n = 7 | 128.6 ± 19.1* |
| INR 3.6 | |
| Control n = 4 | 596.3 ± 39.4 |
| Study 1 n = 8 | 238.1 ± 87.6** |
| INR 5.3 | |
| Control n = 4 | 311.3 ± 83.4 |
| Study 1 n = 8 | 175 ± 21.2*** |

*p < 0.001
**p < 0.0001
***p < 0.04

Human plasma treated with zeolite clotted significantly faster than untreated control plasma independently from the INR level.

### Example 2

### Use of Zeolite to Treat Human Plasma from Patients Having Hemophilia

Human plasma was also obtained from patients diagnosed with Hemophilia A (Factor VIII less than 1%) and Hemophilia B (Factor IX less than 1%). This human plasma was divided into 2 groups (Control and Study 2) and was tested in a modified APTT manual test. In this test, 0.25 ml of plasma was incubated at 37 C. in the presence of 0.025 M CaCl (0.25 ml obtained from Biomerieux, Durham, N.C.) and 0.25 ml Platelet Factor 3 reagent (Partial Thromboplastin) (also obtained from Biomerieux, Durham, N.C.). Zeolite material was added to the Study 2 group samples. Results are shown in Table 2.

TABLE 2
Table 2: Zeolite treated plasma clots significantly faster than
untreated controls.

| | Time to clot: |
|---|---|
| | seconds |
| Hemophilia A | |
| Control n = 15 | 133.8 ± 26.9 |
| Study 2 n = 26 | 106.7 ± 22.1* |
| Hemophilia B | |
| Control n = 12 | 105.2 ± 32.2 |
| Study 2 n = 18 | 84.2 ± 23.2** |

*p < 0.002
**p < 0.05

Human plasma treated with zeolite clotted significantly faster than untreated control plasma for both Hemophilia A and B.

### Example 3

### Use of Zeolite to Treat Human Plasma from Patients Having von Willebrand Disease

Human plasma was obtained from patients affected by von Willebrand disease, both mild (Type I and II) and severe (Type III). The human plasma was divided into 2 groups (Control and Study 3) and was tested in a modified APTT manual test. For this test, 0.25 ml of plasma was incubated at 37 C. in the presence of 0.025 M CaCl (0.25 ml obtained from Biomerieux, Durham, N.C.) and 0.25 ml Platelet Factor 3 reagent (Partial Thromboplastin) (also obtained from Biomerieux, Durham, N.C.). Zeolite material was added to the Study 3 group samples. Results are shown in Table 3.

TABLE 3
Table 3: Zeolite treated plasma clots significantly faster than
untreated controls.

| | Time to clot: |
|---|---|
| | seconds |
| Mild von Willebrand | |
| Control n = 5 | 83.6 ± 5.5 |
| Study 3 n = 19 | 75.6 ± 5.7* |
| Severe von Willebrand | |
| Control n = 8 | 124.1 ± 15.4 |
| Study 3 n = 13 | 109.5 ± 19.8** |

*p < 0.01
**p < 0.01

Human plasma treated with zeolite clotted significantly faster than untreated control plasma for both forms of von Willebrand disease.

### Example 4

### Hemostatic Efficacy of Kaolin-Impregnated Gauze on Anti-Coagulated Animal Subjects

The scope of this experiment was to show that kaolin-impregnated gauze is effective in rapidly stopping bleeding in patients undergoing an anti-coagulation therapy (e.g., being treated with Coumadin® or Plavix® (a brand of clopidogrel)).

In this experiment, a total of 10 pigs were divided into two groups. The animals in the first group (n=5) were treated with Coumadin® and underwent PT testing that included INR measurement. In this testing, INR above 2.5 was targeted. Once PT testing showed that the INR was in the targeted range (greater than 2.5), the animals were prepared for surgery.

The animals in the second group (n=5) were treated with Plavix® according to a dosage typically recommended for humans. Analysis of medical literature indicated that the same dosage was usually used for pigs.

Animals from both groups underwent a series of surgical tests to evaluate the ability of a kaolin-impregnated gauze hemostatic device to control bleeding in anti-coagulated hosts when compared to standard surgical control gauze. Under general anesthesia, the animals underwent a midline laparotomy wherein the peritoneal cavity was entered. The animals then underwent a series of bleeding injuries to the spleen, liver, and mesentery.

The injuries that were treated with either kaolin-impregnated gauze or control standard surgical gauze were:

| | Coumadin ® | Plavix ® |
|---|---|---|
| Splenic injuries | n = 33 | n = 35 |
| Hepatic injuries | n = 16 | n = 20 |
| Mesenteric | n = 37 | n = 35 |

Following the onset of bleeding, manual pressure was held for five minutes. The wound was then observed for bleeding. Blood saturation of the gauze was also evaluated. Failure was defined as persistent bleeding at five minutes, and success was defined as bleeding being stopped completely at five minutes. Failure was also declared when brisk bleeding was noticed during the five minutes during which manual pressure was applied, the gauze became completely soaked with blood, and a determination was made that the animal had become or could have become unstable.

In addition, femoral vessels (both arterial and venous) were surgically exposed by bilateral groin dissection. Animals then underwent bilateral transaction of both femoral artery and vein, and kaolin-impregnated gauze was then immediately applied. (For this portion of the experiment, n=7 for Coumadin® and n=4 for Plavix®.) Manual pressure was held for five minutes after which the wound was observed for re-bleeding. Control gauze was not tested in this set of experiments since literature clearly shows that standard surgical gauze is not effective in controlling this level of severe bleeding.

The data collected was compared by chi-square statistical analysis. A value of p<0.05 was considered significant.

In Group 1, the pigs treated with Coumadin® clearly showed that kaolin-impregnated gauze is significantly more successful in stopping bleeding than standard control surgical gauze. In 90 total injuries, kaolin-impregnated gauze successfully controlled bleeding in 95% of cases as opposed to 24% of cases for the control surgical gauze (p<0.0001).

Similarly, in Group 2, the pigs treated with Plavix® clearly show that kaolin-impregnated gauze is significantly more successful in stopping bleeding than standard control surgical gauze. In 94 total injuries, kaolin-impregnated gauze successfully controlled bleeding in 91% of cases as opposed to 30% for control surgical gauze (p<0.0001).

In conclusion, extensive testing in vivo shows that kaolin-impregnated gauze is highly effective in controlling bleeding in the presence of anti-coagulation of blood following treatment with Coumadin® (or other warfarin-type drugs) or Plavix®.

In taking into account the results of each of the above Examples, it can be concluded that both zeolite and clay (such as kaolin or other layered clay), when used individually, clot human plasma faster than untreated controls in the following conditions: patients treated with Coumadin® (INR1.9, 3.6, 5.3), patients treated with Plavix®, patients affected by Hemophilia A (Factor VIII less than about 1%), and patients affected by Hemophilia B (Factor IX less than about 1%). In addition, zeolites and clays clot human plasma faster than untreated controls in patients affected by von Willebrand disease both mild and severe.

Although this invention has been shown and described with respect to the detailed embodiments thereof, it will be understood by those of skill in the art that various changes may be made and equivalents may be substituted for elements thereof without departing from the scope of the invention. In addition, modifications may be made to adapt a particular situation or material to the teachings of the invention without departing from the essential scope thereof. Therefore, it is intended that the invention not be limited to the particular embodiments disclosed in the above detailed description, but that the invention will include all embodiments falling within the scope of the appended claims.

What is claimed is:

1. A method of clotting blood, said method comprising the step of:
administering a therapeutically effective amount of a composition comprising clay as the active ingredient to a wound from which said blood emanates;
wherein said blood has a compromised ability to form clots.

2. The method of claim 1, wherein said blood includes a coumarin-based composition as an anticoagulant composition.

3. The method of claim 1, wherein said blood includes warfarin.

4. The method of claim 1, wherein said blood is obtained from a patient diagnosed with Hemophilia A.

5. The method of claim 1, wherein said blood is obtained from a patient diagnosed with Hemophilia B.

6. The method of claim 1, wherein said blood is obtained from a patient diagnosed with von Willebrand disease.

7. The method of claim 1, wherein said blood includes a clopidogrel-based composition as an anticoagulant composition.

8. The method of claim 1, wherein said clay is kaolin.

9. The method of claim 1, wherein said clay is a layered clay.

10. A method of arresting blood flowing from a wound, said method comprising the steps of:
providing a patient being inflicted with a bleeding wound; and
administering a therapeutically effective amount of a composition comprising clay as the active ingredient to said bleeding wound;
wherein said blood has a compromised ability to form clots.

11. The method of claim 10, wherein said patient is treated with a coumarin-based composition as an anticoagulant composition.

12. The method of claim 10, wherein said patient is treated with warfarin.

13. The method of claim 10, wherein said blood is obtained from a patient diagnosed with Hemophilia A.

14. The method of claim 10, wherein said blood is obtained from a patient diagnosed with Hemophilia B.

15. The method of claim 10, wherein said blood is obtained from a patient diagnosed with von Willebrand disease.

16. The method of claim 10, wherein said step of administering said therapeutically effective amount of said composition comprises placing said composition directly on said bleeding wound.

17. The method of claim 10, wherein said patient is treated with a clopidogrel-based composition as an anticoagulant composition.

18. The method of claim 10, wherein said clay is kaolin.

19. The method of claim 10, wherein said clay is a layered clay.

20. The method of claim 10, wherein said step of administering said therapeutically effective amount of said composition comprises placing said composition on bandages and placing said bandages directly on said bleeding wound.
